(19) United States
(12) Patent Application Publication
(10) Pub. No.: US 2009/0171346 A1
(43) Pub. Date: Jul. 2, 2009

(54) HIGH CONDUCTIVITY INDUCTIVELY EQUALIZED ELECTRODES AND METHODS

(76) Inventor: Greg Leyh, Brisbane, CA (US)

Correspondence Address: Cutera c/o MoFo, 425 Market Street, San Francisco, CA, 94105-2482, US

(21) Appl. No.: 11/966,895

(22) Filed: Dec. 28, 2007

(51) Int. Cl. A61B 18/14

(52) U.S. Cl. 606/35

(57) ABSTRACT

Apparatus and methods for evenly distributing electric current density over a surface of at least one of an active electrode and a return electrode during electrosurgery, wherein the active and/or return electrode includes a spiral inductor. The spiral inductor may include a low electrical resistivity material or a spiral bare metal surface for contacting the patient's body. In a multi-layer spiral inductor having a plurality of stacked spirals, each turn of a first spiral may be electrically coupled in series to a radially corresponding turn of each successive one of the stacked spirals; and each turn of the innermost spiral may be electrically coupled to an adjacent, radially outward turn of the outermost spiral.

### FIELD OF THE INVENTION

The present invention generally relates to apparatus and methods for electrosurgery.

### BACKGROUND OF THE INVENTION

Various forms of electrosurgery are now widely used for a vast range of surgical procedures. There are two basic forms or electrosurgery, namely monopolar and bipolar, according to the configuration of the electrosurgical system which determines the path of electrical energy flow vis-à-vis the patient. In the bipolar configuration, both the active electrode and the return electrode are located adjacent to a target tissue of the patient, i.e., the electrodes are in close proximity to each other, and current flows between the electrodes locally at the surgical site.

In monopolar electrosurgery, the active electrode is again located at the surgical site; however, the return electrode, which is typically much larger than the active electrode, is placed in contact with the patient at a location on the patient's body that is remote from the surgical site. Current from an electrosurgical generator typically flows through an active electrode and into target tissue of the patient. The current then passes through the patient's body to the return electrode where it is collected and returned to the generator. In monopolar electrosurgery, the return electrode is typically accommodated on a device which may be referred to as a dispersive pad, and the return electrode may also be known as the dispersive-, patient-, neutral-, or grounding electrode. In general, monopolar electrosurgical procedures allow a large range of tissue effects.

A disadvantage of monopolar electrosurgery using prior art return electrodes is the risk of burns on the patient's body at the location of the return electrode. In the case of a solid return electrode, e.g., a metal plate or sheet, electric current density tends to be concentrated at the corners and/or edges of the return electrode. Concentration, or uneven distribution, of electric current density at the return electrode surface may cause excessive heating to the extent that a severe burn to the patient's tissue can result.

One approach to solving the problem of return electrode-induced patient burns has been to use multiple dispersive pads. However, with the increase in the number of dispersive pads, the correct placement becomes more difficult, while incorrect placement of the pads also increases the risk of a patient burn. Increasing the number of dispersive pads may also complicate monitoring of dispersive pad contact with the patient.

In an attempt to reduce edge effects and the uneven distribution of electric current density, U.S. Pat. No. 5,836,942 to Isaacson discloses a biomedical electrode having one or two conductive plates and a field of lossy dielectric material disposed between the plate(s) and the patient. U.S. Pat. No. 7,169,145 also to Isaacson discloses a return electrode that is self-limiting and self-regulating as to maximum current and temperature rise. An inductor coupled in series with the electrode counteracts at least a portion of the impedance of the return electrode and the patient to optimize current flow when the contact area of the electrode on the patient is sufficient to perform electrosurgery.

U.S. Patent Application Publication No. 20060074411 (Carmel et al.) discloses a dispersive electrode in which an intermediate layer of conductive dielectric is disposed between the conducting component(s) and the patient. Carmel et al. discloses various configurations, including various spiral or pseudo-spiral configurations, for the conducting component(s), and the conductive dielectric may be disposed on both sides of the conducting component(s). The conductive dielectric disposed between the conducting component(s) and the patient uses self-resistance for resistive dispersion of electric current density over the return pad.

A similar disadvantage of monopolar electrosurgery, using prior art active electrodes for treating a target tissue, is uneven electric current density distribution over the surface of the active electrode, e.g., current density may be concentrated at the corners and/or edges of the active electrode. Such uneven distribution of electric current density over the active electrode surface may lead to uneven heating or treatment of the patient's tissue with undesirable effects on the patient.

As can be seen, there is a need for apparatus and methods for safely performing monopolar electrosurgery using a return electrode that prevents patient burns. There is a further need for apparatus and methods for electrosurgical treatment of a patient using an active electrode that prevents uneven treatment of the patient's tissue.

### SUMMARY OF THE INVENTION

According to one aspect of the invention, there is provided apparatus comprising an electrosurgical instrument including an active electrode unit. The active electrode unit comprises at least one spiral inductor, each spiral inductor includes a spiral comprising an electrically conductive metal, and each spiral inductor is configured for applying electrical energy to a target tissue of the patient's body. According to another aspect of the invention, there is provided apparatus for receiving electrical energy from a patient. The apparatus comprises a dispersive return pad including a return electrode unit. The return electrode unit comprises at least one spiral inductor, and each spiral inductor includes at least one spiral comprising an electrically conductive metal. The spiral inductor is configured for contacting a patient's body. The return electrode unit includes a patient-contacting surface, and the patient-contacting surface comprises either a patient-contacting layer having an electrical resistivity value less than 0.1 Ohm.m disposed on the spiral inductor, or a bare metal surface of the spiral inductor.

According to a further aspect of the invention, a method for treating a patient comprises disposing an active electrode unit in relation to a target tissue of the patient's body, wherein the active electrode unit comprises at least one spiral inductor; and applying electrical energy, via the spiral inductor, to the target tissue.

According to still another aspect of the invention, there is provided a method for performing electrosurgery on a patient, the method comprising contacting the patient's body with a return electrode unit, wherein the return electrode unit comprises a spiral inductor; applying electrical energy to the patient's body via an active electrode unit, wherein the active electrode unit is coupled to a power supply; and receiving the electrical energy from the patient's body via the spiral inductor. The return electrode unit includes a patient-contacting surface. The spiral inductor comprises an electrically conductive metal, and the patient-contacting surface comprises either a patient-contacting layer having an electrical resistivity value less than 0.1 Ohm.m disposed on the spiral inductor, or a bare metal surface of the spiral inductor.

According to yet another aspect of the invention, a method for making a multi-layer spiral inductor comprises forming a plurality of spirals, wherein each spiral comprises an electrically conductive metal disposed on an electrically insulating support layer; stacking the plurality of spirals; electrically coupling, in series, each turn of a first spiral of the plurality of spirals to a radially corresponding turn of each successive one of the plurality of spirals; and electrically coupling each turn of an innermost spiral of the plurality of spirals to an adjacent, radially outward turn of the first or outermost spiral, with the proviso that a radially outermost turn of the innermost spiral is not coupled to an adjacent, radially outward turn of the first spiral.

These and other features, aspects, and advantages of the present invention may be further understood with reference to the drawings, description, and claims which follow.

### BRIEF DESCRIPTION OF THE DRAWINGS

FIG. 1 is a block diagram schematically representing electrosurgical apparatus, according to an embodiment of the invention;

FIG. 2 is a block diagram schematically representing electrosurgical apparatus including an active electrode unit having a spiral inductor, according to another embodiment of the invention;

FIG. 3 is a block diagram schematically representing electrosurgical apparatus including a return electrode unit having a spiral inductor, according to another embodiment of the invention;

FIG. 4A schematically represents a spiral for a spiral inductor, as seen in plan view, according to another embodiment of the invention;

FIG. 4B schematically represents a spiral of a spiral inductor having a variable pitch, as seen in plan view, according to another embodiment of the invention;

FIG. 4C schematically represents a spiral of a spiral inductor having a variable pitch, as seen in plan view, according to another embodiment of the invention;

FIG. 5 schematically represents a multi-layer spiral inductor, as seen in side view, according to another embodiment of the invention;

FIG. 6A schematically represents a spiral inductor, including a plurality of vertically stacked spirals, having electrical connections between turns of each spiral, as seen in side view, according to another embodiment of the invention;

FIG. 6B schematically represents a multi-layer spiral inductor, including a plurality of vertically stacked spirals, showing connections between turns of each spiral, as seen in side view, according to another embodiment of the invention;

FIG. 7A schematically represents a spiral inductor having a substantially circular or oval configuration, as seen in plan view, according to another embodiment of the invention;

FIG. 7B schematically represents a spiral inductor having a substantially square or rectangular configuration, as seen in plan view, according to another embodiment of the invention;

FIG. 8 is a block diagram schematically representing an electrosurgical instrument including an active electrode unit having a spiral inductor, according to an embodiment of the invention;

FIG. 9A schematically represents a spiral inductor for an active electrode unit, as seen in plan view, according to an embodiment of the invention;

FIG. 9B schematically represents a spiral inductor for an active electrode unit, as seen in side view, according to another embodiment of the invention;

FIG. 9C schematically represents a multi-layer spiral inductor including a plurality of vertically stacked spirals, as seen in side view, according to an embodiment of the invention;

FIG. 10A schematically represents an active electrode unit including a treatment face defined by a plurality of co-planar spiral inductors, as seen in plan view, according to another embodiment of the invention;

FIG. 10B schematically represents the active electrode unit of FIG. 10A, as seen in perspective view, according to another embodiment of the invention;

FIG. 11 schematically represents an electrosurgical instrument including a plurality of spiral inductors, according to another embodiment of the invention;

FIG. 12A schematically represents an active electrode unit, or portion thereof, including a plurality of spiral inductors, as seen in plan view, according to another embodiment of the invention;

FIG. 12B schematically represents an active electrode unit, or portion thereof, including a plurality of spiral inductors, as seen in plan view, according to another embodiment of the invention;

FIG. 13A schematically represents a return electrode unit including a spiral inductor, as seen in plan view, according to an embodiment of the invention;

FIG. 13B schematically represents a dispersive return pad including a spiral inductor having a bare metal patient-contacting surface, as seen in side view, according to an embodiment of the invention;

FIG. 13C schematically represents a dispersive return pad including a spiral inductor having a low electrical resistivity patient-contacting layer thereon, as seen in side view, according to another embodiment of the invention;

FIG. 13D schematically represents a dispersive return pad including a multi-layer spiral inductor, as seen in side view, according to another embodiment of the invention;

FIG. 14 schematically represents a monopolar electrosurgical procedure for treating a patient using at least one of an active spiral inductor and a return spiral inductor, according to an embodiment of the invention;

FIG. 15A is a flow chart schematically representing steps in a method for treating a patient using an active spiral inductor, according to another embodiment of the invention;

FIG. 15B is a flow chart schematically representing steps in a method for treating a patient using an active spiral inductor, according to another embodiment of the invention;

FIG. 16 is a flow chart schematically representing steps in a method for performing electrosurgery using a return spiral inductor, according to another embodiment of the invention;

FIG. 17A is a flow chart schematically representing steps in a method for making a spiral inductor, according to another embodiment of the invention; and

FIG. 17B is a flow chart schematically representing steps in a method for electrically coupling a plurality of spirals for a multi-layer spiral inductor, according to another embodiment of the invention.

### DETAILED DESCRIPTION OF THE INVENTION

The following detailed description is of the best currently contemplated modes of carrying out the invention. The description is not to be taken in a limiting sense, but is made merely for the purpose of illustrating the general principles of the invention, since the scope of the invention is best defined by the appended claims.

Broadly, the present invention provides methods and apparatus for performing monopolar electrosurgical procedures in a safe and effective manner while preventing the uneven treatment of a target tissue and/or patient burns. Patient burns are known to occur using apparatus and methods of the prior art due to uneven distribution of electric current density over the surface of conventional return electrodes. In contrast to prior art devices, return electrode units of the instant invention are configured for evenly distributing electric current density thereover, thereby preventing patient burns. The present invention may also permit higher total current density at the return electrode, and, for a given procedure/electric power usage, the use of a return electrode unit having a smaller patient-contacting area as compared with conventional return electrodes. The present invention may also permit the use of fewer return pads (e.g., a single return pad) for a given procedure/electric power usage, as compared with prior art procedures using a larger number of conventional return pads.

In one aspect, the invention provides apparatus and methods for performing electrosurgery on a patient, wherein a return electrode unit of the apparatus includes at least one spiral inductor. In another aspect, the invention provides apparatus and methods for treating a target tissue of a patient's body, wherein an active electrode unit of the apparatus includes at least one spiral inductor. In yet another aspect of the invention, both the active electrode unit and the return electrode unit may include one or more spiral inductors.

The methods and apparatus of the instant invention may find many applications, including a broad range of monopolar electrosurgical procedures and other biomedical procedures. Such procedures may involve, for example, without limitation: cutting and/or coagulation during general surgery, as well as various cosmetic procedures, and the like.

Some prior art electrosurgical return electrodes have used a field of lossy dielectric material disposed between the electrode(s) and the patient, or a positive temperature coefficient (PTC) material on the electrode surface, to prevent edge effects (which may cause patient burns). Other prior art return electrodes have used one or more electrodes coupled to a central conducting plate via resistive and/or capacitive elements to provide voltage distribution. Still other prior art return electrodes have used an intermediate layer of conductive dielectric, disposed between conducting elements and the patient, for voltage distribution.

Unlike electrosurgical return electrodes of the prior art, in an embodiment the present invention provides apparatus including a return electrode unit including at least one spiral inductor having a sufficiently large number of turns, such that the electric current density at the spiral inductor of the return electrode unit may be evenly distributed thereover. The return electrode unit may include a patient-contacting surface, and in one embodiment, the patient-contacting surface may comprise a patient-contacting layer having an electrical resistivity value less than 0.1 Ohm.m disposed on the spiral inductor. In another embodiment, the patient-contacting surface may comprise a bare metal surface of the spiral inductor.

In another embodiment, and in contrast to active electrodes of the prior art, the present invention provides apparatus including an active electrode unit having at least one spiral inductor having a sufficiently large number of turns, such that the electric current density at the spiral inductor of the active electrode unit may be evenly distributed thereover. Advantageously, even electric current density distribution provided by apparatus and methods of the instant invention may prevent the uneven heating of treated tissue thereby increasing the efficacy of treatment as well as patient safety, as compared with prior art devices and methods. Furthermore, heating of tissue via spiral inductors of the present invention may obviate the need for actively cooling target or non-target tissue during treatment.

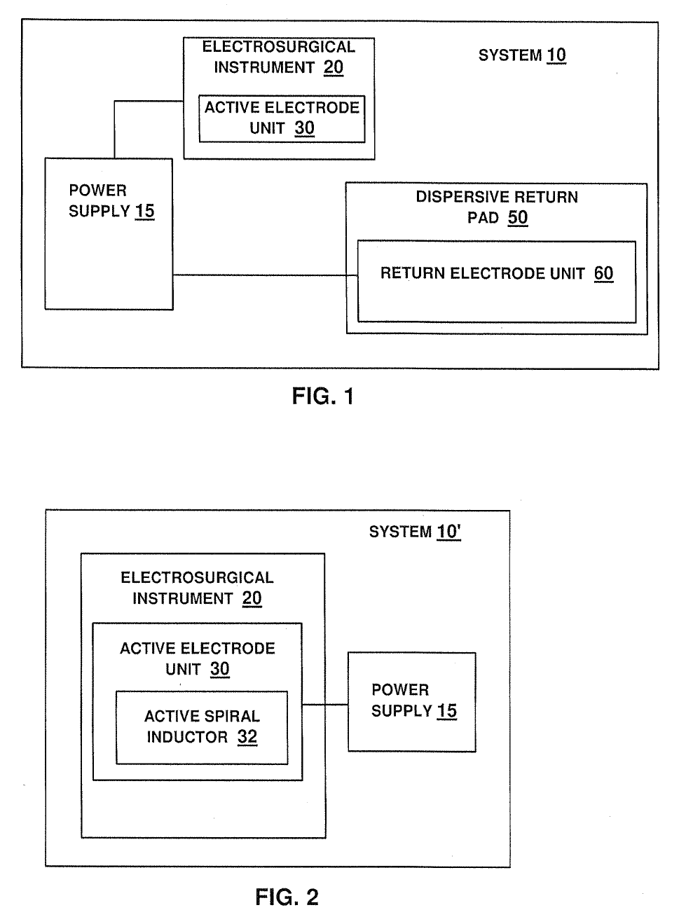

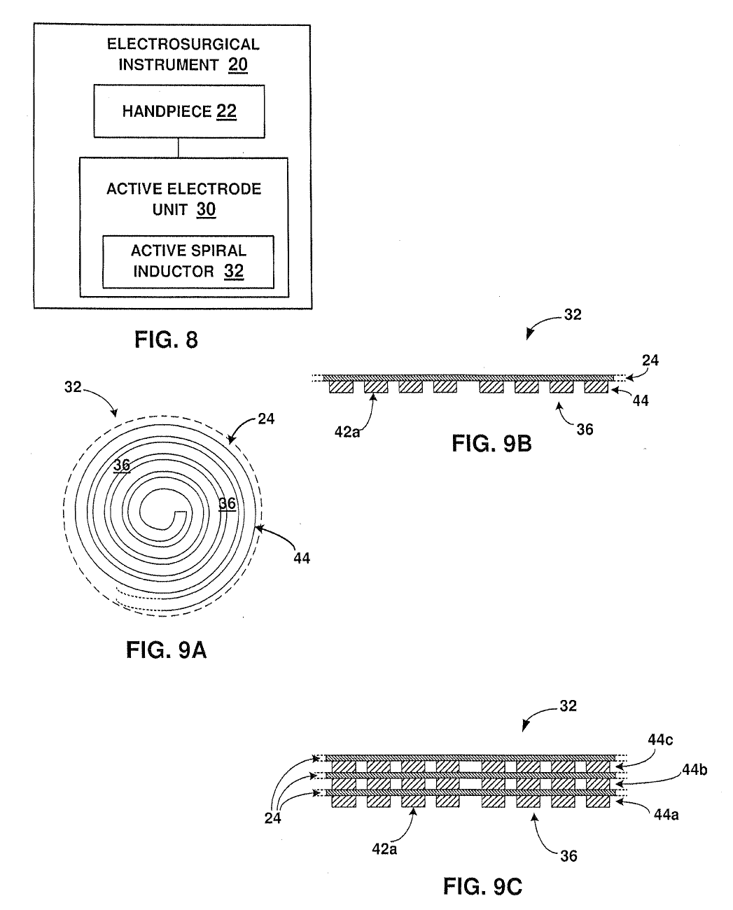

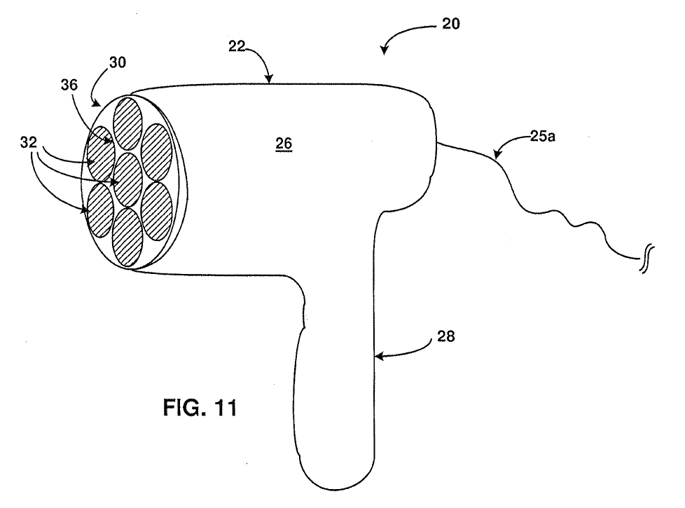

FIG. 1 is a block diagram schematically representing electrosurgical apparatus, according to an embodiment of the invention. Electrosurgical system 10 of FIG. 1 may include an electrosurgical generator or power supply 15, an electrosurgical instrument 20, and a dispersive return pad 50. Electrosurgical system 10 may be configured for monopolar electrosurgery. Power supply 15 may be configured for supplying electrical energy, such as radiofrequency (RF) alternating current, to electrosurgical instrument 20. Electrosurgical instrument 20 may be configured for electrical coupling to power supply 15, and for applying electrical energy to a patient's body or tissue(s) during a procedure. Embodiments of an electrosurgical instrument 20 are schematically represented hereinbelow (see, e.g., FIGS. 8 and 11, infra). Dispersive return pad 50 may include a return electrode unit 60. Dispersive return pad 50 may be configured for promoting contact between return electrode unit 60 and a patient's body.

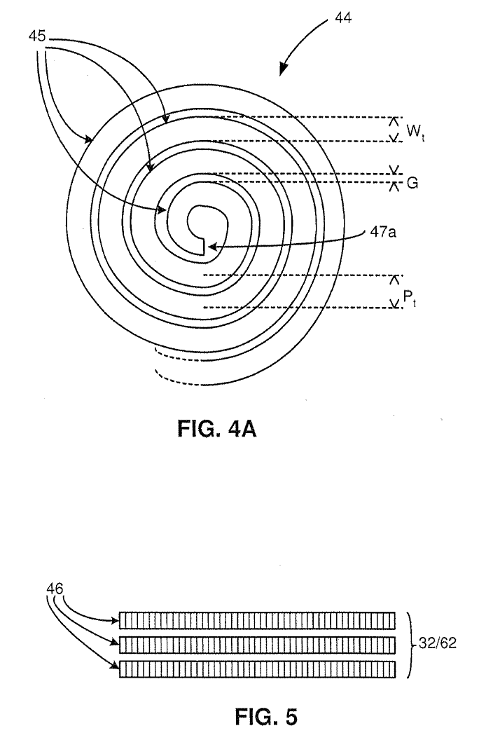

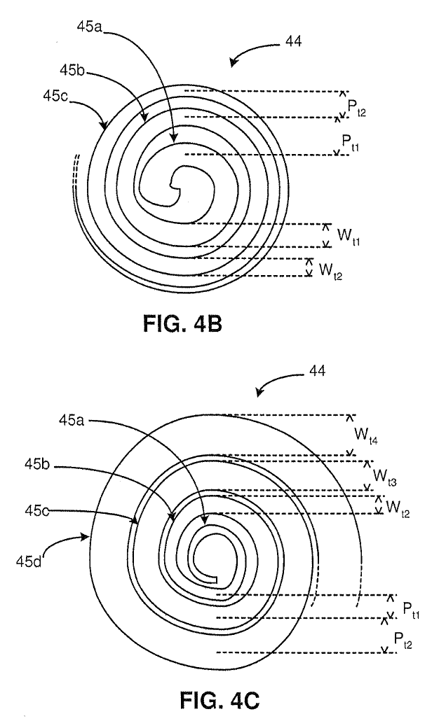

FIG. 2 is a block diagram schematically representing electrosurgical apparatus, according to another embodiment of the invention. Electrosurgical system 10′ of FIG. 2 may include an electrosurgical instrument 20 having an active electrode unit 30. Active electrode unit 30 may be configured for electrical coupling to power supply 15. Active electrode unit 30 may include at least one spiral inductor, which may be referred to herein as an active spiral inductor 32. Active spiral inductor(s) 32 may be configured for applying electrical energy to a patient's body (see, for example, FIG. 11). Active spiral inductor 32 may have suitable self-inductance for promoting the even distribution of electrical current density thereover while active electrode unit 60 is applying electrical energy to the patient's body during a procedure. Active spiral inductor 32 may comprise one or more spirals of electrically conductive metal (see, e.g., FIGS. 4A-C, 5, 6A-B, and 9C).

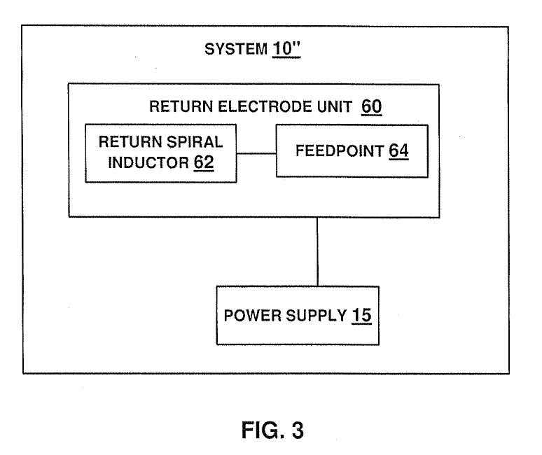

FIG. 3 is a block diagram schematically representing electrosurgical apparatus, according to an embodiment of the invention. Electrosurgical system 10″ of FIG. 3 may include a return electrode unit 60 and a power supply 15. Return electrode unit 60 may include a spiral inductor, which may be referred to herein as a return spiral inductor 62, and a feedpoint 64 electrically coupled to return spiral inductor 62. In an embodiment, return spiral inductor 62 may comprise a plurality of spirals of electrically conductive metal, wherein the plurality of spirals are stacked and electrically interconnected (see, for example, FIG. 4A-C, 5, 6A-B, and 13D).

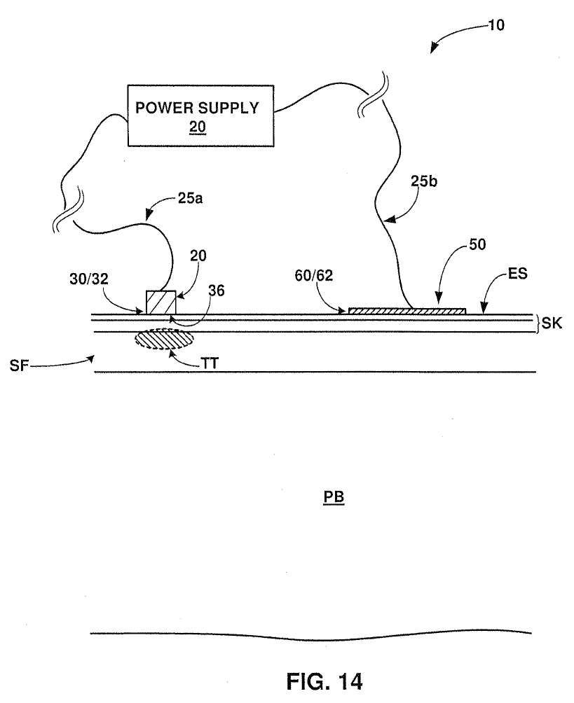

Return spiral inductor 62 may be configured for contacting a patient's body (see, for example, FIG. 14). Return spiral inductor 62 may have suitable self-inductance for promoting the even distribution of electrical current density thereover while return electrode unit 60 is receiving electrical energy from the patient's body during a procedure.

### Electrically Conductive Spirals and Spiral Inductors

There now follows a description of electrically conductive spirals and spiral inductors that may be used in a broad range of applications.

FIG. 4A schematically represents a spiral of electrically conductive material, as seen in plan view, according to another embodiment of the invention. Spiral 44 may include a plurality of turns 45 and an inner terminus 47a. Only a few of the radially inner turns of spiral 44 are shown in FIG. 4A, whereas spiral 44 may comprise from about 10 to 200 or more turns, typically from about 20 to 150 turns, often from about 30 to 150 turns, and usually from about 40 to 120 turns. As an example, spiral 44 may comprise a spiral trace of an electrically conductive metal, such as Cu, Al, or various alloys, as non-limiting examples. In an embodiment, spiral 44 may comprise a filament of the electrically conductive metal, wherein the filament may be disposed on a support layer 24. In an embodiment, spiral 44 may be formed (e.g. onto a substrate) by a printing process or a printing-like process.

As shown in FIG. 4A, spiral 44 may have a pitch, Pt, representing a radial distance between the radial midpoints of adjacent turns 45. The pitch of spiral 44 may be in the range of from about 0.1 mm to 10 mm or more, typically from about 0.2 mm to 9 mm, often from about 0.25 to 5 mm, and in some embodiments from about 0.3 to 1.5 mm. In an embodiment, the pitch of spiral 44 may be constant or substantially constant. In other embodiments, the pitch of spiral 44 may vary (see, e.g., FIGS. 4B-C).

Turns 45 of spiral 44 may have a width, Wₜ, wherein the width, Wₜ is a radial distance across each turn 45. The width of each of turns 45 may typically be in the range of from about 0.05 mm to 10 mm or more, typically from about 0.15 to 9 mm, often from about 0.2 to 5 mm, and in some embodiments from about 0.25 to 1.5 mm. In an embodiment, the width of the various turns 45 may be constant or substantially constant. In other embodiments, the width of turns 45 may vary (see, e.g., FIGS. 4B-C). A profile or cross-sectional shape of turns 45 may be substantially rectangular or rounded; typically the width of each turn 45 may be greater than its height.

A gap, G may exist between adjacent turns 45 of spiral 44, wherein the gap may represent a radial distance between opposing edges of adjacent turns 45. The gap is typically less than the pitch, usually the gap is substantially less than the pitch, and often the gap is considerably less than the pitch. The gap between turns 45 of spiral 44 may typically be in the range of from about 0.1 mm to 0.5 mm, usually from about 0.15 to 0.4 mm, and often from about 0.15 to 0.3 mm. In an embodiment, the gap between adjacent turns 45 may be constant or substantially constant, even though the pitch may be variable (see, e.g., FIGS. 4B-C). The gap between turns 45 may be air, as a non-limiting example.

FIG. 4B schematically represents a spiral 44 of electrically conductive material, as seen in plan view, according to another embodiment of the invention. As shown in FIG. 4B, spiral 44 may have a variable pitch, wherein the pitch (shown as Pₜ₁, Pₜ₂) may increase in a radially inward direction. For example, in the embodiment of FIG. 4B the following relationship may exist: Pₜ₁>Pₜ₂. As also shown in FIG. 4B, turns 45 of spiral 44 may have a variable width, Wₜ wherein the width of first and second turns 45a, 45b, respectively (shown as Wₜ₁, Wₜ₂) may also increase in a radially inward direction, wherein Wₜ₁>Wₜ₂.

FIG. 4C schematically represents a spiral 44 of electrically conductive material, as seen in plan view, according to another embodiment of the invention. As shown in FIG. 4C, spiral 44 may have a variable pitch, wherein the pitch (shown as Pₜ₁, Pₜ₂) may increase in a radially outward direction. For example, in the embodiment of FIG. 4C the following relationship may exist: Pₜ₁<Pₜ₂. As also shown in FIG. 4C, turns 45 of spiral 44 may have a variable width, Wₜ wherein the width (shown as Wₜ₂, Wₜ₃, Wₜ₄) may also increase in a radially outward direction, wherein Wₜ₂<Wₜ₃<Wₜ₄.

With further reference to FIGS. 4B-C, in an embodiment wherein the pitch of spiral 44 may be variable (i.e., the pitch may increase or decrease in a radial direction), the width of the turns, the pitch, and the gap between opposing edges of adjacent turns, may be substantially as described hereinabove with reference to FIG. 4A. In various embodiments of the invention, the pitch of spiral 44 may be variable over all or part of spiral 44, wherein the pitch over all or part of spiral 44 may increase or decrease in a radial direction according to either a continuous or discontinuous gradient. In an embodiment, the variation in pitch and width between adjacent turns 45 of spiral 44 may extend over 150 or more turns 45 of spiral 44.

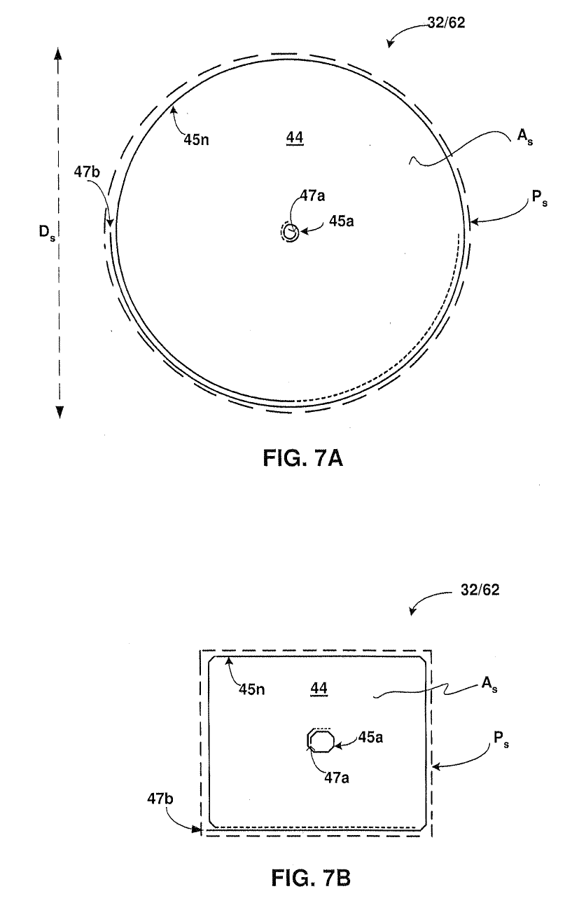

Spiral 44 of the invention may be at least substantially planar. Coils of spiral 44 may be laterally or radially spaced-apart. Spirals 44 of the invention may be configured such that the width of a given turn of spiral 44 is much greater than the gap between that turn and an adjacent turn (see, e.g., FIG. 4A). Therefore, according to an aspect of the present invention, most of the external surface area of a spiral inductor 32/62 formed by spiral 44 may be occupied by electrically conductive metal of spiral 44 (see, e.g., FIGS. 7A-B). Although spirals 44 of FIGS. 4A-C are shown as being at least substantially circular in configuration, other configurations including oval, square, rectangular, and the like, are also within the scope of the invention. In a square or rectangular configuration of spiral 44, acute angles and right angles may be avoided; for example, in some embodiments spiral 44 may have obtuse angles (see, e.g., FIG. 7B).

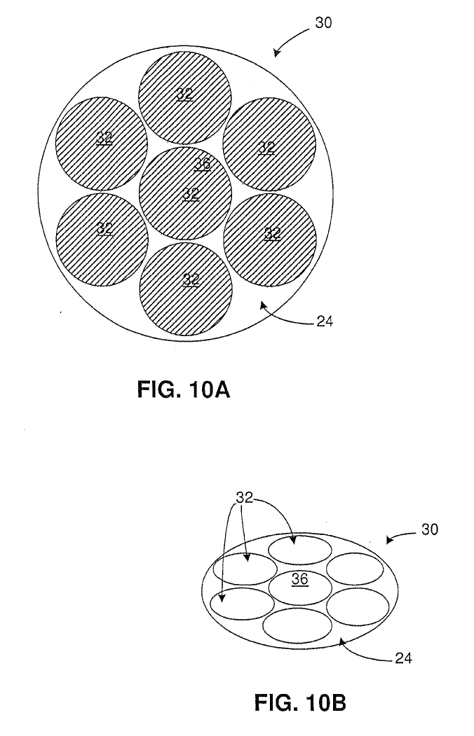

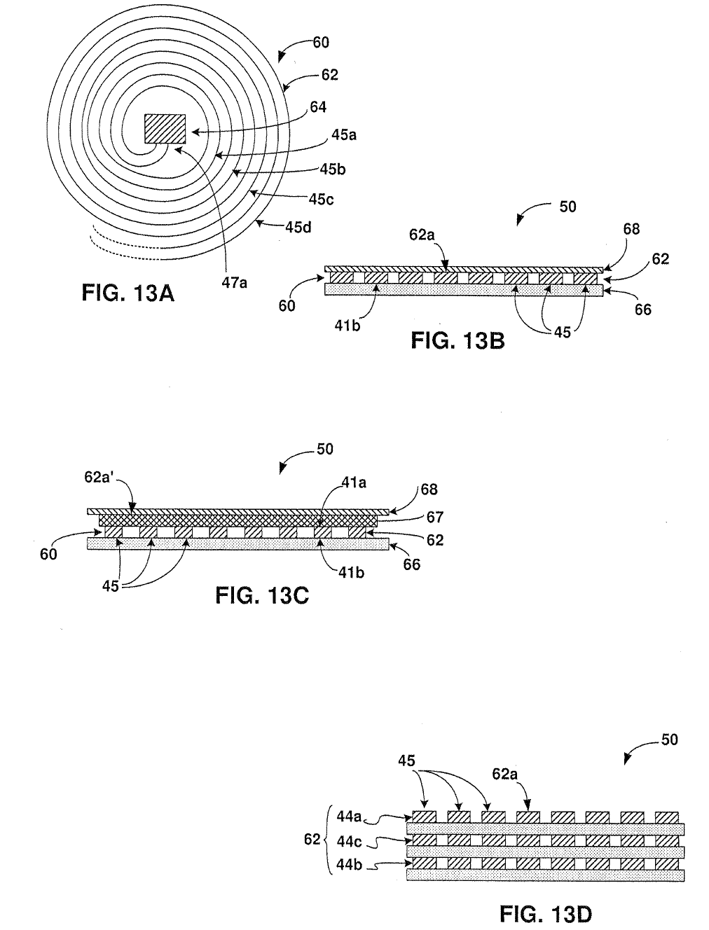

FIG. 5 schematically represents a multi-layer spiral inductor having a plurality of vertically stacked electrically conductive spirals, as seen in side view, according to another embodiment of the invention. As shown, spiral inductor 32/62 may include three, vertically stacked spiral layers 46. Each of spiral layers 46 may include a spiral 44 of electrically conductive metal (see, e.g., FIG. 4A), wherein each spiral 44 may be disposed on a support layer (not shown). Spiral inductor 32/62 may comprise an active spiral inductor 32 for an active electrode unit 30 (see, e.g., FIGS. 9A-C), or a return spiral inductor 62 for a return electrode unit 60 (see, e.g., FIGS. 13B-D).

Although three layers are shown in FIG. 5, other numbers of layers are also within the scope of the invention. Typically, spiral inductor 32/62 may include about two (2) to four (4) spiral layers. In general, the more spiral layers, the greater the inductive effect per unit area of spiral inductor 32/62.

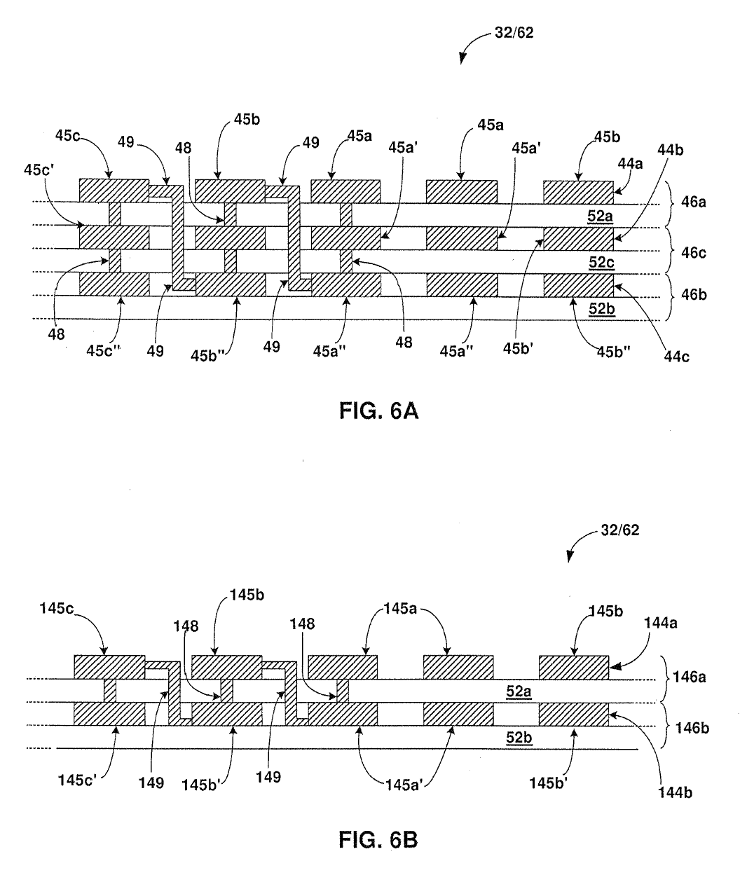

FIG. 6A schematically represents a central portion of a multi-layer spiral inductor, as seen in side view, according to another embodiment of the invention. Spiral inductor 32/62 may be a component of an active electrode unit 30 or a return electrode unit 60. Spiral inductor 32/62 may include a first or outermost spiral layer 46a, an innermost spiral layer 46b, and at least one intermediate spiral layer 46c. For each spiral 44a, 44b, and 44c, only a first, a second, and a third turn 45a, 45b, 45c, respectively, are shown in FIG. 6A for the sake of clarity, it being understood that each spiral 44a, 44b, and 44c may comprise from about 20 to 150 or more turns. Turns of spirals 44a, 44b, and 44c, including first, second, and third turns 45a, 45b, 45c, as well as additional turns not shown in FIG. 6A, may be generally referred to as turns 45 (see, e.g., FIG. 4A).

Again with reference to FIG. 6A, first or outermost spiral layer 46a may be defined as a layer of spiral inductor 32/62 that is closest to, or in contact with, the patient's body during use of spiral inductor 32/62 (e.g., as a component of active electrode unit 30 or return electrode unit 60). In some embodiments, intermediate layer 46c may represent one or more spiral layers, although only a single intermediate layer 46c is shown in FIG. 6A. In another embodiment, intermediate layer 46c may be omitted to provide a two-layer spiral inductor (see, for example, FIG. 6B). Each layer of spiral inductor 32/62, e.g., outermost layer 46a, innermost layer 46b, and intermediate layer 46c, may comprise spiral 44a, spiral 44b, and spiral 44c, respectively.

With further reference to FIG. 6A, spirals 44a-c may be referred to as a first or outermost spiral 44a, a second or intermediate spiral 44b, and an innermost spiral 44c, respectively. Each spiral 44a, 44b, and 44c may comprise an electrically conductive metal, for example as a metal trace or filament. Spirals 44a, 44b, and 44c may each have the same spiral configuration, e.g., each spiral 44a-c may have the same number of turns, the same pitch, the same trace width, and the same gap width, etc. In an embodiment, spirals 44a, 44b, and 44c may be stacked vertically such that radially corresponding turns of each of spirals 44a, 44b, and 44c are aligned with each other. Spirals 44a, 44b, and 44c may be disposed on a first or outermost support layer 52a, an innermost support layer 52b, and an intermediate support layer 52c, respectively.

With still further reference to FIG. 6A, turns 45 of spirals 44a, 44b, and 44c may be electrically coupled in the following manner: each turn, e.g., first turn 45a, of first spiral 44a may be electrically coupled, in series, to a radially corresponding turn of each successive spiral, i.e., turns 45a′ and 45a″ of spirals 44b and 44c; and, each turn of innermost spiral 44c, e.g., turn 45a″, may be electrically coupled to an adjacent, radially outward turn of first (outermost) spiral 44a, i.e., turn 45b. An exception to this pattern of connection may exist for the radially outermost turn of innermost spiral 44c, since the radially outermost turn lacks an adjacent radially outward turn (e.g., as can be seen from FIG. 6A, turn 45c″ could not be coupled to an adjacent, radially outward turn of first spiral 44a, since there is no turn located radially outward from turn 45c″).

The same manner of interconnection as described with reference to FIG. 6A may be used for other numbers of vertically stacked spirals 44, each having any number of turns 45. Each turn 45 may be electrically coupled, in series, to a radially corresponding turn of each successive spiral by vertical connections 48, while each turn of innermost spiral 44c may be electrically coupled to an adjacent, radially outward turn of outermost spiral 44a by radial connections 49. In this regard, all radially corresponding turns of adjacent spiral layers may be interconnected by vertical connections 48, whereas radial connections 49 only couple radially non-corresponding turns of innermost and outermost spirals 46b, 46a, respectively.

For the embodiment of FIG. 6A, the interconnection of turns 45 of spiral layers 46a-c to provide a three-layer spiral inductor may be described more specifically as follows:
1) first turn 45a of the first spiral 44a may be electrically coupled to a first turn 45a′ of second spiral 44b,
2) first turn 45a′ of second spiral 44b may be electrically coupled to a first turn 45a″ of third spiral 44c,
3) first turn 45a″ of third spiral 44c may be electrically coupled to a second turn 45b of first spiral 44a,
4) second turn 45b of first spiral 44a may be electrically coupled to a second turn 45b′ of second spiral 44b,
5) second turn 45b′ of second spiral 44b may be electrically coupled to a second turn 45b″ of third spiral 44c, and
6) second turn 45b″ of third spiral 44c may be electrically coupled to a third turn 45c of first spiral 44a, etc. Thus, first turn 45a, 45a′, 45a″ of first through third spirals 44a-c, respectively, may jointly define a first set of turns of spiral inductor 32/62; each of a plurality of successive sets of turns of first through third spirals 44a-c may be coupled to each other in series; and each turn 45 of third spiral 44c may be coupled to an adjacent radially outward turn of first spiral 44a. As noted hereinabove, an exception to this connection pattern may exist for the radially outermost turn of third spiral 44c, which naturally lacks a radially outward turn. It is to be understood that the coupling between specific turns enumerated hereinabove may be performed in sequences other than as listed to provide a multi-layer spiral inductor having turns electrically coupled as shown in FIGS. 6A-B.

In describing the manner of interconnectivity of turns 45 for the embodiment of FIG. 6A, first turn 45a, 45a′, 45a″ of first, second, and third spirals 44a-c, respectively, may represent the radially innermost turn of the first, second, and third spirals 44a-c, respectively; first, second, and third spirals 44a, 44b, and 44c may be vertically stacked on top of each other. First spiral 44a may occupy first or outermost spiral layer 46a; and third spiral 44c may occupy innermost spiral layer 46b (see, FIG. 6A).

For purposes of illustration, each spiral 44a, 44b, and 44c is shown in FIG. 6A as having first, second, and third turns 45a, 45b, 45c, respectively, wherein first turn 45a may be located substantially centrally with respect to each spiral 44a, 44b, and 44c. In practice, each spiral 44a, 44b, and 44c may comprise from about 10 to 200 turns, typically from about 20 to 150 turns, often from about 30 to 150 turns, and usually from about 40 to 120 turns. However, the manner of interconnecting turns of spirals 44a, 44b, and 44c may be as shown in FIG. 6A regardless of the number of turns in each spiral. Namely, each turn, e.g., turn 45a, of first spiral 44a may be electrically coupled, in series, to a radially corresponding turn (turns 45b, 45c) of successive spirals 44c, 44b; and each turn 45 of innermost spiral 44c may be electrically coupled to an adjacent, radially outward turn 45 of first spiral 44a, with the proviso (as noted above) that a radially outermost turn of innermost spiral 44c is not so coupled to an adjacent radially outward turn of first spiral 44a.

FIG. 6B schematically represents a central portion of a multi-layer spiral inductor 32/62, including two stacked spirals, according to another embodiment of the invention. Spiral inductor 32/62 of FIG. 6B may include a first or outermost spiral 144a and a second or innermost spiral 144b. Turns of first and second spirals 144a, 144b including first and second turns 145a, 145b, as well as additional turns not shown in FIG. 6B, may be referred to herein generically as turns “45” (see, e.g., FIG. 4A). In the spiral inductor 32/62 of FIG. 6B, turns 45 of spirals 144a, 144b may be interconnected between layers 46a and 46b as follows:
1) first turn 145a of first spiral 144a may be electrically coupled to a first turn 145a′ of second spiral 144b,
2) first turn 145a ′ of second spiral 144b may be electrically coupled to a second turn 145b of first spiral 144a,
3) second turn 145b of first spiral 144a may be electrically coupled to a second turn 145b ′ of second spiral 144b, and
4) second turn 145b ′ of second spiral 144b may be electrically coupled to a third turn 145c of first spiral 144a, etc. It is to be understood that the coupling between specific turns enumerated hereinabove may be performed in sequences other than as listed to provide a multi-layer spiral inductor having turns electrically coupled as shown in FIGS. 6A-B.

With further reference to FIG. 6B, radially corresponding turns of first and second spirals 144a, 144b may be interconnected by vertical connections 148, while connection between turns of second spiral 144b and a radially outer turn of first spiral 144a (i.e., between radially non-corresponding turns) may be by radial connections 149. First turn 145a, 145a′ of first and second spirals 144a, 144b, respectively, may jointly define a first set of turns of spiral inductor 32/62. Each of a plurality of successive sets of turns of first and second spirals 144a, 144b may be electrically coupled to each other, and each turn of second spiral 144b may be coupled to an adjacent radially outward turn of first spiral 144a, with the proviso that the radially outermost turn of second spiral 144b lacks an adjacent radially outward turn. It can be seen that the interconnection of turns 45 of the two-layer spiral inductor 32/62 of FIG. 6B follows the same general pattern of electrical coupling as for the embodiment of FIG. 6A.

FIG. 7A schematically represents a spiral inductor, as seen in plan view, according to another embodiment of the invention. Spiral inductor 32/62 of FIG. 7A may have a substantially circular or oval configuration. Spiral inductor 32/62 may include a spiral trace 44 of electrically conductive metal having an inner terminus 47a and an outer terminus 47b. For clarity, sections of the spiral trace 44 that are between the terminuses are not shown in FIG. 7A. Spiral inductor 32/62 may include a plurality of turns, from a first turn 45a (radially innermost) to an n^{th} turn 45n (radially outermost). In an embodiment, n may be from about 10 to 200 or more, substantially as described hereinabove. Spiral inductor 32/62 may have a perimeter, Pₛ, and an external surface area Aₛ defined by the perimeter. The electrically conductive metal of spiral 44 may occupy at least about 50% of a total surface area Aₛ, that is to say, at least about 50 percent (%) of the external surface area of spiral inductor 32/62 may be occupied by spiral 44. Typically, electrically conductive metal of spiral 44 may occupy from about 60 to 99% of external surface area, Aₛ; usually from about 70 to 99% of external surface area, Aₛ; often from about 75 to 98% of external surface area, Aₛ; and in some embodiments electrically conductive metal of spiral 44 may occupy from about 85% to 97% of external surface area, Aₛ. Spiral 44 may have a diameter, Dₛ, typically in the range of from about 20 to 0.1 cm, usually from about 12 to 0.2 cm, and often from about 10 to 0.4 cm.

FIG. 7B schematically represents a spiral inductor, according to another embodiment of the invention. Spiral inductor 32/62 may include a spiral trace 44 of electrically conductive metal having an inner terminus 47a, an outer terminus 47b, and a plurality of turns, 45a-n, substantially as described for the embodiment of FIG. 7A. For clarity, sections of the spiral trace 44 that are between the terminuses are not shown in FIG. 7B. Spiral inductor 32/62 of FIG. 7B may have a substantially square or rectangular configuration, a perimeter, Pₛ, and a surface area Aₛ defined by the perimeter. Spiral inductor 32/62 may include a spiral trace 44 of electrically conductive metal. Spiral trace 44 may occupy a percentage of surface area, As generally as described with reference to FIG. 7A.

In an embodiment, spiral inductors 32/62 of FIGS. 7A-B may comprise a single spiral 44 which may be at least substantially planar. In another embodiment, spiral inductors 32/62 of FIGS. 7A-B may comprise a plurality of vertically stacked spirals 44, wherein each of the plurality of spirals 44 may be at least substantially planar.

### Spiral Inductors for Active Electrode Applications

FIG. 8 is a block diagram schematically representing an electrosurgical instrument, according to another embodiment of the invention. Electrosurgical instrument 20 may include a handpiece 22 and an active electrode unit 30. Active electrode unit 30 may include an active spiral inductor 32. Electrosurgical instrument 20 may be coupled to power supply 15 (see, e.g., FIG. 2) to form apparatus configured for the application of electrical energy, via spiral inductor 32, to a target tissue of a patient. Electrosurgical instrument 20, active electrode unit 30, and active spiral inductor 32 may have various other features, elements, and characteristics substantially as described herein for various embodiments of the invention.

FIG. 9A schematically represents a spiral inductor for an active electrode unit, as seen in plan view, according to another embodiment of the invention. Active spiral inductor 32 may comprise an electrically conductive metal spiral 44 (see, e.g., FIGS. 4A-C). As an example, spiral 44 may comprise a spiral trace of electrically conductive metal, such as Cu, Al, or various alloys. In an embodiment, spiral 44 may comprise a filament of the electrically conductive metal. In an embodiment, spiral 44 may be formed by a printing process or a printing-like process. An external surface 42a of spiral 44 may define a treatment face 36 of spiral inductor 32 and active electrode unit 30.

Only a radially inner portion of spiral 44 is shown in FIG. 9A, whereas spiral 44 in its entirety may include many more turns. For example, in an embodiment spiral 44 may have from about 10 to 200 turns, typically 20 to 150 turns, often from about 30 to 150 turns, and usually from about 40 to 120 turns. Spiral 44 may have a variable or constant pitch between adjacent turns (see, e.g., FIGS. 4A-C).

Spiral 44 may be disposed on a support layer 24. Support layer 24 may comprise an electrically insulating or dielectric material. Examples include, but are not limited to, Teflon, Polyamide, FR4, G10, Nylon, Polyester, Kapton, Silicone, or Rubber. In an embodiment, support layer 24 may be at least substantially equivalent to one of support layers 52a-c (see, FIGS. 6A-B). In use, spiral 44 may be disposed between support layer 24 and the patient's body. Active spiral inductor 32 may be configured for evenly distributing electric current density thereover via self-inductance of spiral 44. Active spiral inductor 32 may be configured for selectively heating a target tissue of the patient's body and for providing a tissue-altering effect on the target tissue.

FIG. 9B schematically represents a portion of a spiral inductor 32 for an active electrode, as seen in side view, according to an embodiment of the invention. (In comparison with FIG. 9A, which shows spiral 44 disposed on top of support layer 24, FIG. 9B is shown as being inverted.) Spiral inductor 32 may be at least substantially planar. In an embodiment, spiral inductor 32 may comprise a spiral 44. Spiral 44 may include an external surface 42a. External surface 42a may be a bare metal surface of electrically conductive metal spiral 44. External surface 42a of spiral 44 may define a treatment face 36. External surface 42a and treatment face 36 may be configured for contacting a patient's body (see, e.g., FIG. 14). Treatment face 36 may be at least substantially planar.

FIG. 9C schematically represents a multi-layer spiral inductor for an active electrode unit, as seen in side view, according to an embodiment of the invention. As shown, active spiral inductor 32 may include a plurality of vertically stacked spirals 44a-c. Spiral 44a may be an outermost spiral 44, while spiral 44c may be referred to as an innermost spiral. Spiral 44b may be referred to as an intermediate spiral. In use, spiral 44a may be closest to, or in contact with a patient's body, while spiral 44c may be the furthest from the patient's body. Each spiral 44a-c may be disposed on a corresponding support layer 24. An external surface 42a of outermost spiral 44a may define a treatment face 36 of active spiral inductor 32. Other numbers of spiral layers 46a-c are also within the scope of the invention.

FIG. 10A schematically represents an active electrode unit, as seen in plan view, and FIG. 10B shows the active electrode unit of FIG. 10A in perspective view, according to another embodiment of the invention. Active electrode unit 30 may include a plurality of active spiral inductors 32. Active spiral inductors 32 may be at least substantially co-planar, or horizontally arranged, on support layer 24. The external surface 42a (see, e.g., FIG. 9B) of the plurality of spiral inductors 32 may jointly define a treatment face 36. Treatment face 36 may be at least substantially planar. Treatment face 36 may be configured for contacting a patient's body, and for applying electrically energy to a target tissue of the patient's body. Active electrode unit 30 may be coupled to power supply 15 to provide an electrosurgical apparatus configured for independently energizing each of spiral inductors 32 of active electrode unit 30. Active electrode unit 30 and power supply 15 may be configured for sequentially energizing spiral inductors 32. Each of the sequentially energized spiral inductors 32 may be energized for various time periods. In an embodiment, a sequence and/or period of energization of spiral inductors 32 may be based on a temperature-related feedback mechanism.

As shown in FIG. 10A, each active spiral inductor 32 may be substantially circular in configuration; however, other configurations are also within the scope of the invention. Although active electrode unit 30 is shown as having seven (7) active spiral inductors 32, other numbers and arrangements of active spiral inductors 32 are also within the scope of the invention.

FIG. 11 schematically represents an electrosurgical instrument, according to another embodiment of the invention. Electrosurgical instrument 20 may include a handpiece 22 and an active electrode unit 30. Active electrode unit 30 may include a plurality of spiral inductors 32. Active spiral inductors 32 may be at least substantially co-planar, such that an external surface 42a of spiral inductors 32 may jointly define a treatment face 36. A cord or cable 25a may be coupled to handpiece 22 for electrically coupling active electrode unit 30 to a power supply (see, e.g., FIGS. 1, 2, and 14). Handpiece 22 may include a housing 26 having a handle 28. Handpiece 22 may be grasped by handle 28 for guiding or moving active spiral inductors 32 and treatment face 36 relative to a treatment area of a patient's body, skin, or target tissue to be treated by electrosurgical instrument 20. Active electrode unit 30 of FIG. 11 may have other features and elements substantially as described with reference to FIGS. 10A-B. Other configurations for handpiece 22, including housing 26 and handle 28, are also within the scope of the invention.

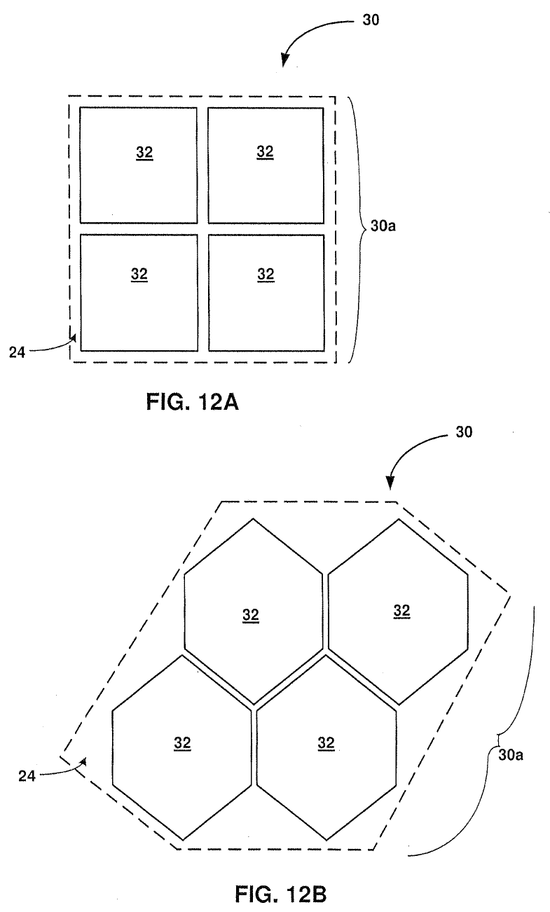

FIG. 12A schematically represents at least a portion of an active electrode unit, according to another embodiment of the invention. Active electrode unit 30 may include a plurality of at least substantially co-planar spiral inductors 32. Spiral inductors 32 may be arranged in the form of an array 30a. Spiral inductors 32 may be disposed on support layer 24. In an embodiment, each of spiral inductors 32 in array 30a may be of equal size, such that spiral inductors 32 may be closely arranged in a regular manner within array 30a. However, other arrangements for array 30a are also within the scope of the invention. In the embodiment of FIG. 12A, each of spiral inductors 32 may be substantially square or rectangular.

FIG. 12B schematically represents at least a portion of an active electrode unit, as seen in plan view, according to another embodiment of the invention. Active electrode unit 30 of FIG. 12B may include a plurality of at least substantially co-planar spiral inductors 32 forming an array 30a. As shown in FIG. 12B, each of spiral inductors 32 may be hexagonal; however, other configurations, such as triangular, octagonal, and the like are also within the scope of the invention.

Active electrode unit 30 and spiral inductors 32 of FIGS. 12A and 12B may have features and elements substantially as described with reference to FIGS. 10A-B. Although FIGS. 12A-B each show active electrode unit 30 as comprising an array 30a having 4 spiral inductors 32, other numbers of spiral inductors 32 are also within the scope of the invention. For example, array 30a of active electrode unit 30 may typically comprise from about 2 to 12 spiral inductors 32, usually from about 2 to 10 spiral inductors 32, and often from about 4 to 8 spiral inductors 32.

### Spiral Inductors for Return Electrode Applcations

FIG. 13A schematically represents a portion of a return electrode unit, as seen in plan view, according to another embodiment of the invention. Return electrode unit 60 may include a return spiral inductor 62. Spiral inductor 62 may comprise a spiral 44 of electrically conductive metal, wherein spiral 44 may have elements and features substantially as described with reference to FIGS. 4A-C. Return electrode unit 60 may further include a feedpoint 64. A spiral inner terminus 47a may be electrically coupled to feedpoint 64.

Feedpoint 64 may be configured for electrically coupling return spiral inductor 62 to power supply 15 (see, e.g., FIG. 3). Spiral inductor 62 may include a plurality of turns, of which only the radially innermost turns, namely first, second, third, and fourth turns 45a, 45b, 45c, 45d, are shown for the sake of clarity. In practice, spiral inductor 62 may comprise from about 10 turns to 200 or more turns, typically from about 20 to 150 turns, often from about 30 to 150 turns, and usually from about 40 to 120 turns. In an embodiment, spiral 44 may be formed by a printing process or a printing-like process. Although spiral inductor 62 is shown in FIG. 13A as being at least substantially circular in configuration, other configurations including oval, square, hexagonal, rectangular, and the like, are also within the scope of the invention (see, e.g., FIGS. 7B, 12A-B).

FIG. 13B schematically represents a dispersive return pad, as seen in side view, according to an embodiment of the invention. Dispersive return pad 50 of FIG. 13B may include a support layer 66, and a return spiral inductor 62 disposed on support layer 66. Spiral inductor 62 may comprise an electrically conductive spiral 44 (see, e.g., FIGS. 4A-C, 13A). Spiral inductor 62 may function as, or be a component of, a return electrode unit 60 (see, e.g., FIGS. 1, 3, and 13A). Support layer 66 may comprise an electrically non-conductive or electrically insulating material. In an embodiment, support layer 66 may be at least substantially planar and flexible or conformable, for example, as in a plastic sheet, or the like. In an embodiment, support layer 66 may be at least substantially equivalent to one of support layers 52a-c (see, FIGS. 6A-B).

As shown in FIG. 13B, spiral inductor 62 may include a patient-contacting surface 62a, wherein patient-contacting surface 62a may comprise a bare metal surface of spiral inductor 62, and such a bare metal patient-contacting surface 62a may be configured for directly contacting the patient's body during a procedure. That is to say, in the embodiment of FIG. 13B, all or part of patient-contacting surface 62a of return electrode unit 60 may be devoid of an adhesive layer, a gel layer, and the like, or any other material; and dispersive return pad 50 may be configured for bare metal contact of return spiral inductor 62 on the patient's body (for example, skin or other tissue). An electrically conductive material, such as an amorphous gel and the like may be applied to the patient's skin, e.g., prior to placement of patient-contacting surface 62a thereon.

Spiral inductor 62 may comprise a spiral metal trace or a metal filament, or the like. Spiral inductor 62 of FIG. 13B may otherwise have various characteristics, features and elements as described, for example, with reference to FIGS. 4A-C. Dispersive return pad 50 may still further include a protective layer 68, which may protect spiral inductor 62 or other components of dispersive return pad 50 during transportation or storage thereof. Naturally, protective layer 68 may be removed and discarded prior to use of dispersive return pad 50.

FIG. 13C schematically represents a dispersive return pad, as seen in side view, according to an embodiment of the invention. Dispersive return pad 50 of FIG. 13C may include a support layer 66, a return spiral inductor 62 disposed on support layer 66, and a patient-contacting layer 67 disposed on return spiral inductor 62. Spiral inductor 62 may function as, or be a component of, a return electrode unit 60. Return electrode unit 60 may have elements and features as described hereinabove, e.g., with reference to FIGS. 1, 3, and 13A. Spiral inductor 62 may comprise a spiral metal trace or a metal filament, and may have various other characteristics, features and elements as described, for example, with reference to FIGS. 4A-C.

With further reference to FIG. 13C, patient-contacting layer 67 may comprise an electrically conductive or low resistivity material. In an embodiment, patient-contacting layer 67 may be specifically selected so as to have a low or very low electrical resistivity. For example, patient-contacting layer 67 may be selected to have a specific resistivity value of <0.1 Ohm.m, typically a specific resistivity value of 0.01 Ohm.m or less, usually a specific resistivity value of 0.001 Ohm.m or less, and preferably a specific resistivity value of 0.0001 Ohm.m or less. In an embodiment, patient-contacting layer 67 may have a specific resistivity value in the range of from about 0.00001 to 0.00000001 Ohm.m or less. In the embodiment of FIG. 13C, an outer portion of patient-contacting layer 67 may define a patient-contacting surface 62a′ of return spiral inductor 62.

In an embodiment, patient-contacting layer 67 may optionally include an adhesive component, for example, a polyacrylate- or polyolefin-based pressure-sensitive adhesive, or a hydrogel adhesive. In an embodiment, patient-contacting layer 67 may be aligned or flush with the perimeter of return spiral inductor 62. Patient-contacting layer 67 may be an amorphous material. Dispersive return pad 50 of FIG. 13C may further include a protective layer 68, which may be disposed on patient-contacting layer 67. Protective layer 68 may protect components of dispersive return pad 50 prior to use of dispersive return pad 50. Protective layer 68 may be configured for facile removal thereof prior to use of dispersive return pad 50.

FIG. 13D schematically represents a dispersive pad including a multi-layer spiral inductor, as seen in side view, according to another embodiment of the invention. In the embodiment of FIG. 13D, dispersive return pad 50 may include a return spiral inductor 62, which may comprise a plurality of spirals 44a-c. Spirals 44a-c may be vertically stacked together. An external surface of outermost spiral 44a may define a patient-contacting surface 62a. Turns 45 of spirals 44a-c may be electrically coupled or interconnected in a specifically defined manner (see, e.g., FIGS. 6A-B) such that the combined self-inductance of the plurality of spirals 44a-c may be maximized per unit area of patient-contacting surface 62a.

Dispersive return pads 50 of the invention, such as those of FIGS. 13B-D, may be configured to provide even distribution of electric current density over spiral inductor 62 during use of dispersive return pad 50. A protective layer 68 (not shown in FIG. 13D) may be disposed on patient-contacting surface 62a. Only the radially inner turns of spirals 44a-c are shown in FIGS. 13B-D. Architectures other than those shown in FIGS. 13B-D for dispersive return pads 50 are also within the scope of the invention.

### Electrosurgical Treatment and Procedures Using Spiral Inductors

FIG. 14 schematically represents a monopolar electrosurgical procedure for treating a patient, according to another embodiment of the invention. Such a procedure may involve placing a dispersive return pad 50 in contact with the patient's body, PB, wherein dispersive return pad 50 may include a return electrode unit 60 (see, e.g., FIGS. 3-4) comprising a spiral inductor 62. Spiral inductor 62 may include at least one spiral 44 of electrically conductive metal, as well as other elements and features as described herein (for example, with reference to FIGS. 4A-C, 6A-B and 7A-B).

As shown, dispersive return pad 50 may be configured for contacting an external surface, ES, of the patient's body, for example, the surface of the skin, SK. Dispersive return pad 50 may be conformable to a non-planar external surface of various parts of the patient's body. Dispersive return pad 50 may be placed in contact with the patient's body via a bare metal patient-contacting surface 62a of spiral inductor 62, or via a patient-contacting surface 62a′ of patient-contacting layer 67 disposed on spiral inductor 62 (see, for example, FIGS. 13B-C).

An electrosurgical instrument 20 and dispersive return pad 50 may be coupled to opposite poles of power supply 15, via cables 25a and 25b, respectively. Power supply 20 may be configured for supplying electrical energy, for example, high frequency (e.g., RF) alternating current, to the patient's body. During the procedure, electrical energy may be applied to the patient's body via electrosurgical instrument 20, and the electrical energy may be received by return electrode unit 60 (see, for example, FIG. 1) of dispersive return pad 50. In an embodiment, return electrode unit 60 may include one or more return spiral inductors 62.

Electrosurgical instrument 20 may include an active electrode unit 30. In an embodiment, active electrode unit 30 may include a spiral inductor 32, wherein an external surface of spiral inductor 32 may define a treatment face 36. Treatment face 36 may be configured for contacting the patient's body and for treating a target tissue, TT, during a procedure. Of course, target tissue(s) other than as specifically shown are also within the scope of the invention.

With further reference to FIG. 14, electrosurgical instrument 20 may be configured for performing various procedures on the patient, which may involve, for example, heating, liquefaction, ablation, etc. of a target tissue of the patient. In a non-limiting example, electrosurgical instrument 20 may be configured for treating the skin or subcutaneous tissues of the patient, e.g., during various aesthetic procedures. In another non-limiting example, electrosurgical instrument 20 may be configured for selectively heating a target tissue of the patient in a non-invasive manner to provide a tissue-altering effect on the target tissue.

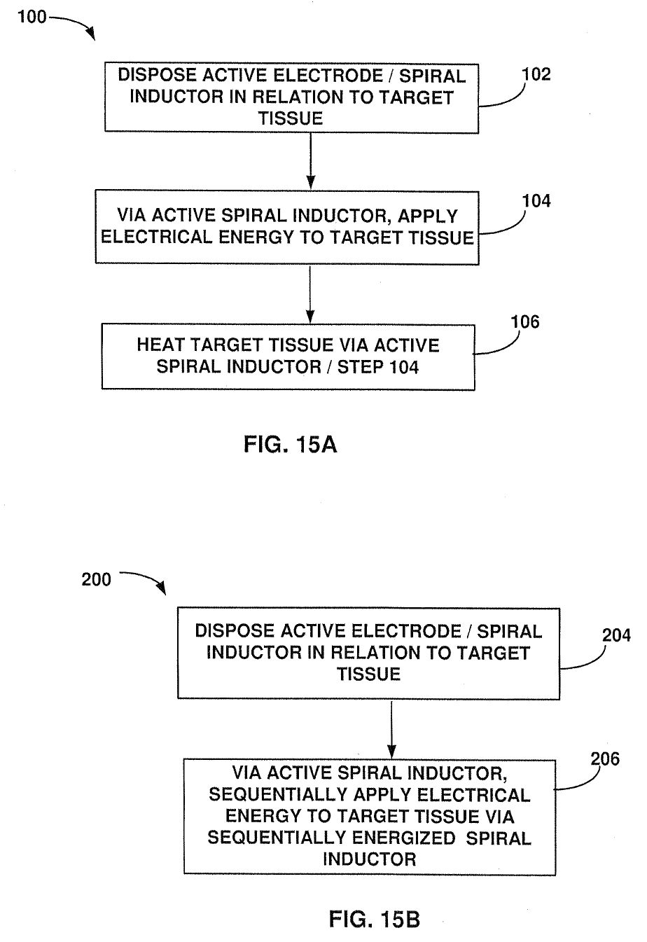

FIG. 15A is a flow chart schematically representing steps in a method 100 for treating a target tissue of a patient, according to another embodiment of the invention. Step 102 may involve disposing an active electrode unit in relation to a target tissue of the patient's body. The active electrode unit may include at least one active spiral inductor configured for evenly applying electrical energy to the target tissue in a treatment area of the patient. Each spiral inductor may comprise at least one spiral of electrically conductive metal. The active electrode unit, spiral inductor(s), and spiral(s) may have various elements, features, and characteristics as described herein with respect to various embodiments of the invention (see, e.g., FIGS. 4A-12B). In an embodiment, at least about 50% of the external surface area of each spiral may be occupied by the electrically conductive metal of spiral 44. Typically, electrically conductive metal may occupy from about 60 to 99% of the external surface area of each spiral; usually from about 70 to 99%; often from about 75 to 98%; and in some embodiments from about 85% to 97%.

In an embodiment, an external surface of the active spiral inductor(s) may be disposed in contact with the patient's body during step 102. As an example, the active electrode unit and its associated active spiral inductor(s) may be located external to the patient's body, e.g., on the skin, during step 102 for non-invasive treatment of a target tissue. The target tissue may comprise subcutaneous tissue (e.g., fat) disposed beneath the skin. In another example, the target tissue may comprise the patient's skin.

Step 104 may involve applying electrical energy to the target tissue via the at least one active spiral inductor. During step 104, the active electrode unit and spiral inductor may be disposed according to step 102. During step 104, the electrical energy may be evenly distributed over a treatment face defined by the external surface of the active spiral inductor (see, e.g., FIG. 9B).

Step 106 may involve heating the target tissue via electrical energy applied via the active spiral inductor according to step 104. The active spiral inductor may be configured for selectively heating the target tissue of the patient's body.

According to one aspect of the present invention, steps 104 and 106 may involve heating the target tissue in the absence of a step for actively cooling the non-target tissue or the target tissue. As an example, a step for actively cooling the patient's tissue in the treatment area may be omitted due to the configuration of the spiral inductor for even distribution of electric current density thereover, such that passive cooling of tissue (e.g., via blood flow) may be sufficient to prevent unwanted damage to target or non-target tissue.

According to an aspect of the invention, step 106 may involve selectively heating the target tissue, such as subcutaneous fat, whereby the target tissue is heated to a higher temperature than that of a non-target tissue, e.g., the skin of the patient. Such selective heating of the target tissue via the active spiral inductor may provide a tissue-altering effect on the target tissue in the absence of adverse effects on non-target tissue or target tissue.

In an embodiment, the active electrode unit may be moved in relation to regions of the target tissue to be treated during the procedure. The electrode unit may be affixed to or integral with a handpiece (see, e.g., FIGS. 8, 11, 14). As non-limiting examples, method 100 may be used to non-invasively treat a target tissue, such as skin or subcutaneous fat of the patient.

FIG. 15B is a flow chart schematically representing steps in a method 200 for treating a target tissue of a patient, according to another embodiment of the invention. Step 202 may involve disposing an electrode unit in relation to target tissue of the patient's body, for example, substantially as described for step 102 of method 100 (see, FIG. 15A). The active electrode unit in the embodiment of FIG. 15B may comprise a plurality of at least substantially co-planar active spiral inductors (see, e.g., FIGS. 10A-12B). Each active spiral inductor may comprise a spiral of electrically conductive metal. The active electrode unit may be coupled to a power supply or electrosurgical generator to provide an electrosurgical apparatus configured for independently energizing each of the plurality of spiral inductors of the active electrode unit. The active electrode unit may be a component of a handpiece. The handpiece may include a treatment face configured for placement and/or movement thereof in relation to the patient's body, e.g., skin of the patient. In an example, the target tissue may comprise subcutaneous fat, and step 202 may involve disposing the electrode unit on a non-target tissue, such as the skin, wherein the targeted tissue may be disposed distal to the non-target tissue and the treatment face. Alternatively, the target tissue may comprise skin, and the treatment face may be placed in contact with the skin (epidermis) for treatment of the skin (dermis). The description of step 202 with respect to target and non-target tissue may similarly be applicable to method 100 (supra).

Step 204 may involve sequentially applying electrical energy to the target tissue via the plurality of spiral inductors, wherein the plurality of spiral inductors may be sequentially energized. A sequence of energization of the plurality of spiral inductors may be based on a temperature of a target tissue or non-target tissue in a treatment area of the patient's body. During step 204, the electrical energy may be evenly distributed over the treatment face defined by an external surface of the spiral inductors.

Methods 100 and 200 of FIGS. 15A-B may each involve the use of a return electrode in a monopolar electrosurgical procedure. However, it is to be understood that methods 100 and 200 do not require a return electrode of a particular configuration; e.g., a return electrode used in method 100 or method 200 may or may not include a spiral inductor.

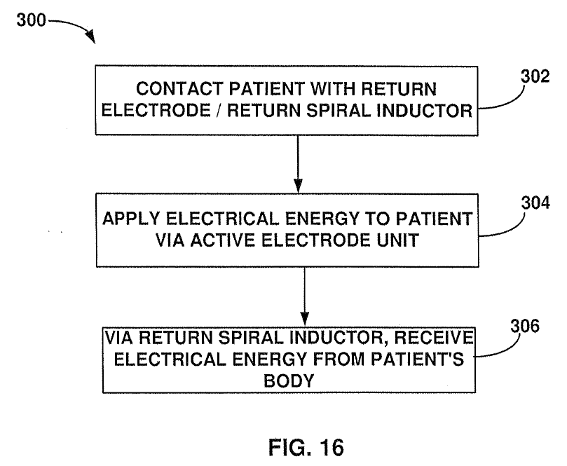

FIG. 16 is a flow chart schematically representing steps in a method for performing electrosurgery on a patient, according to another embodiment of the invention. Step 302 of method 300 may involve contacting a patient's body with a spiral inductor of a return electrode unit. Typically, step 302 may involve contacting an external surface, such as the skin surface, of the patient's body with a patient-contacting surface of the spiral inductor. The return electrode unit and spiral inductor may be affixed to a support layer of a dispersive return pad.

During step 302 the dispersive return pad may be disposed on the patient's body, wherein the dispersive return pad may be configured for promoting contact of a patient-contacting surface of the return spiral inductor with the patient's body. In an embodiment, the patient-contacting surface may comprise a patient-contacting layer comprising a low resistivity material having an electrical resistivity value of less than 0.1 Ohm.m. In another embodiment, step 302 may involve contacting the patient's body with a bare metal surface of at least a portion of the return spiral inductor. Such a bare metal surface may be an external surface of an electrically conductive metal spiral trace. The spiral or spiral trace of electrically conductive metal may be at least substantially planar, and may have elements and features as described hereinabove (see, e.g., FIGS. 4A-C).

Step 304 of method 300 may involve applying electrical energy to the patient via an active electrode unit. The active electrode unit may be a component of an electrosurgical instrument (see, for example, FIG. 14). During step 304, electrical energy may be applied to a target tissue, e.g., skin, adipose tissue, connective tissue, cardiovascular tissue, joint tissue, gastrointestinal tissue, endocrine tissue, nervous tissue, etc., to effect treatment of the patient. It is to be understood that method 300 does not require an active electrode of any particular configuration. For example, an active electrode used in step 304 may or may not include a spiral inductor.

Step 306 may involve receiving the electrical energy, from the patient's body, via the return spiral inductor placed in contact with the patient in step 302. The return spiral inductor may be coupled to a return terminal of the power supply. The return spiral inductor may comprise one or more spirals of electrically conductive metal. In an embodiment, a plurality of such spirals may be stacked vertically and each turn of each spiral may be electrically coupled in a specific sequence, e.g., as described with reference to FIGS. 6A-B, to provide a return spiral inductor configured for dispersing a relatively large amount of electrical energy per unit area of the return electrode unit.

### Methods for Making Spiral Inductors

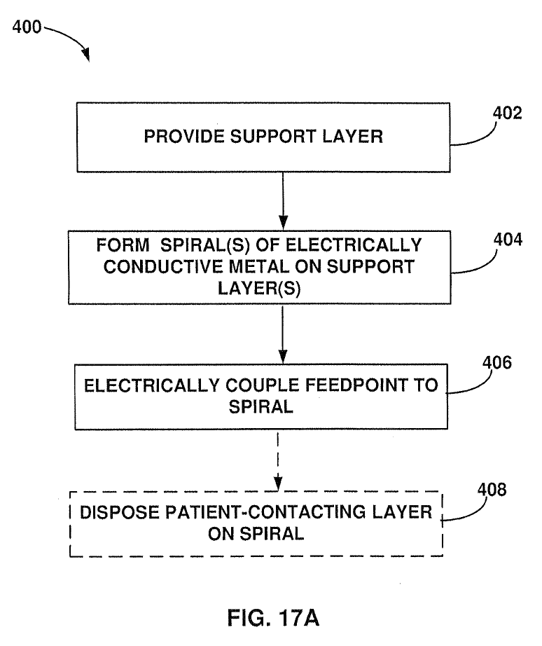

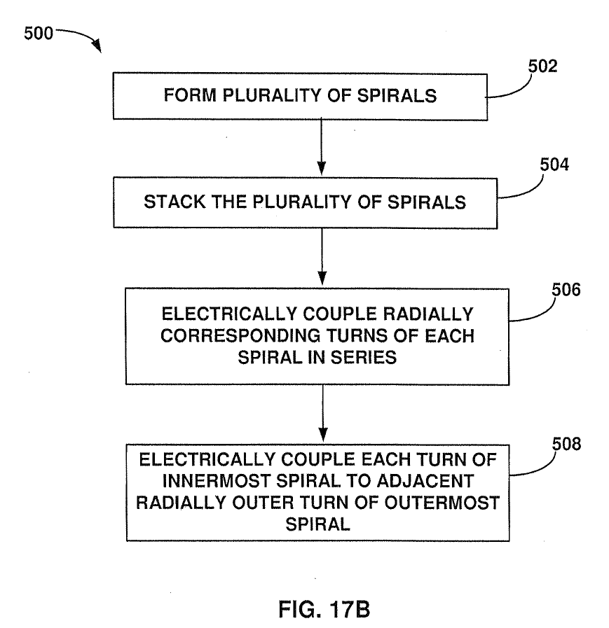

FIG. 17A is a flow chart schematically representing steps in a method 400 for making a spiral inductor for a return electrode unit, according to another embodiment of the invention. Step 402 may involve providing a support layer. The support layer may comprise a layer or sheet of an electrically insulating or non-conductive material.

Step 404 may involve forming at least one spiral of electrically conductive metal on at least one support layer. For example, in embodiments where the spiral inductor includes a plurality of spirals, each spiral of electrically conductive metal may be formed on a separate support layer. A lower portion of each spiral may be in contact with the support layer. Each spiral may be formed as a trace of the electrically conductive metal, or each spiral may be deposited on the support layer as a filament of the electrically conductive metal. In an embodiment, a metal trace forming each spiral may be formed by a printing, or printing-like, process. As a non-limiting example, one or more printing processes similar to those used for the production of flexible electrical circuits may be used in step 404. The spiral(s) formed in step 404 and described elsewhere herein according to the present invention, may be referred to as comprising a metal “trace”, regardless of the techniques or processes for forming such spiral(s). Each spiral may have an inner terminus (see, for example, FIG. 4).

Step 406 may involve electrically coupling an inner terminus of the spiral to a feedpoint. The feedpoint may be configured for coupling the spiral to an electrosurgical power supply. In an embodiment, the spiral may be electrically coupled to one or more additional spirals in a specific manner (see, for example, FIGS. 6A-B, 17B).

An upper portion of the spiral may define a bare metal surface of the spiral, wherein the metal surface may define a patient-contacting surface which may contact the patient's body during a procedure. In some embodiments, optional step 408 may involve disposing a patient-contacting layer on the metal surface of the spiral, such that the patient-contacting layer defines a patient-contacting surface. The patient-contacting layer may comprise an electrically conductive material having an electrical resistivity value less than 0.1 Ohm.m, and in some embodiments 0.01 Ohm.m or less.

FIG. 17B is a flow chart schematically representing steps in a method 500 for electrically coupling a plurality of electrically conductive spirals for forming a multi-layer spiral inductor, according to another embodiment of the invention. Step 502 may involve forming a plurality of spirals of electrically conductive metal. Each of the spirals may be formed generally as described with reference to FIG. 17A. Each of the spirals may have elements and features as described hereinabove, e.g., with reference to one or more of FIGS. 4A-C.

Step 504 may involve stacking the plurality of spirals. The spirals may have identical spiral configurations, essentially as described hereinabove, e.g., with reference to FIG. 6A. The spirals may be stacked vertically, and the plurality of spirals may be aligned with each other.

Steps 506 and 508 may involve electrically coupling the plurality of spirals. The spirals may be interconnected such that each turn of the plurality of spirals is coupled to at least one other spiral. The turns of each spiral may be interconnected, for example, by connections such as vias, or the like. In an embodiment, the spirals may be interconnected in a specific manner, for example, as shown in FIGS. 6A-B. Thus, step 506 may involve electrically coupling, in series, each radially corresponding turn of each spiral. For example, each turn of a first spiral of the plurality of stacked spirals may e coupled to a radially corresponding turn of each successive one of the spirals.

Step 508 may involve electrically coupling each turn of an innermost spiral of the plurality of spirals to an adjacent, radially outward turn of the first or outermost spiral, with the proviso that a radially outermost turn of the innermost spiral is not so coupled to an adjacent, radially outward turn of the first spiral. The interconnection of electrically conductive traces in general, e.g., by various types of vias, is well known in the printed circuit board art, as an example. In various embodiments, step 508 may be performed before or after step 506.

The disclosed systems may be provided with instructions for use instructing the user to use the system in accordance with the disclosed methods.

As may be appreciated by the skilled artisan, methods and apparatus of the invention may find many applications other than those specifically described herein.

It should be understood that the foregoing relates to exemplary embodiments of the invention, none of the examples presented herein are to be construed as limiting the present invention in any way, and that modifications may be made without departing from the spirit and scope of the invention as set forth in the following claims.

What is claimed is:

1. Apparatus for treating a patient, said apparatus comprising:
an electrosurgical instrument including an active electrode unit,
said active electrode unit comprising at least one spiral inductor,
each said spiral inductor including at least one spiral comprising an electrically conductive metal, and
each said spiral inductor is configured for applying electrical energy to a target tissue of the patient's body.

2. The apparatus of claim 1, wherein:
said spiral inductor includes an external surface,
said external surface of said spiral inductor defines a treatment face,
said treatment face is configured for contacting the patient's body, and
said electrically conductive metal of said spiral occupies from about 60 to 99% of the area of said external surface of said spiral inductor.

3. The apparatus of claim 1, wherein:
each said spiral comprises from about 20 to 150 turns, and
said electrically conductive metal of said spiral occupies from about 85 to 97% of the area of said external surface of said spiral inductor.

4. The apparatus of claim 1, wherein:
said spiral inductor comprises a plurality of said spirals of said electrically conductive metal,
said plurality of spirals are stacked vertically, and
each said spiral has the same spiral configuration, wherein:
each turn of a first spiral of said plurality of spirals is electrically coupled in series to a radially corresponding turn of each successive one of said plurality of spirals, and
each turn of an innermost spiral of said plurality of spirals is electrically coupled to an adjacent, radially outward turn of said first spiral, with the proviso that a radially outermost turn of said innermost spiral is not so coupled to an adjacent radially outward turn of said first spiral,
and wherein said first spiral is an outermost spiral of said plurality of spirals.

5. The apparatus of claim 1, wherein:
said active electrode unit comprises a plurality of said spiral inductors,
said plurality of spiral inductors are arranged in an array such that said plurality of spiral inductors are at least substantially co-planar, and
said apparatus is configured for sequentially energizing said plurality of spiral inductors.

6. The apparatus of claim 3, wherein:
said spiral has a pitch in the range of from about 0.25 mm to 5 mm,
each said turn has a width in the range of from about 0.2 mm to 5 mm, and
said spiral inductor is configured for selectively heating the target tissue of the patient's body and for providing a tissue-altering effect on the target tissue.

7. Apparatus for receiving electrical energy from a patient, said apparatus comprising:
a dispersive return pad including a return electrode unit,
said return electrode unit comprising at least one spiral inductor,
each said spiral inductor includes at least one spiral comprising an electrically conductive metal,
said spiral inductor is configured for contacting a patient's body,
said return electrode unit includes a patient-contacting surface, and
said patient-contacting surface comprises:
a patient-contacting layer having an electrical resistivity value less than 0.1 Ohm.m disposed on said spiral inductor, or
a bare metal surface of said spiral inductor.

8. The apparatus of claim 7, further comprising:
a power supply coupled to said return electrode unit; and
an active electrode unit coupled to said power supply, wherein:
said spiral inductor comprises a return spiral inductor, and
said active electrode unit comprises an active spiral inductor.

9. The apparatus of claim 7, wherein:
said at least one spiral has a pitch in the range of from about 0.25 mm to 5 mm, and
each said spiral comprises from about 20 to 150 turns.

10. The apparatus of claim 7, wherein:
said spiral inductor comprises a plurality of said spirals of said electrically conductive metal,
said plurality of spirals are stacked vertically, and
each said spiral has the same spiral configuration, wherein:
each turn of a first spiral of said plurality of spirals is electrically coupled in series to a radially corresponding turn of each successive one of said plurality of spirals, and
each turn of an innermost spiral of said plurality of spirals is electrically coupled to an adjacent, radially outward turn of said first spiral, with the proviso that a radially outermost turn of said innermost spiral is not so coupled to an adjacent radially outward turn of said first spiral,
and wherein said first spiral is an outermost spiral of said plurality of spirals.

11. The apparatus of claim 7, wherein said electrically conductive metal of said spiral occupies from about 75 to 98% of the area of said external surface of said spiral inductor.

12. A method for treating a patient, comprising:
a) disposing an active electrode unit in relation to a target tissue of the patient's body, wherein said active electrode unit comprises at least one spiral inductor; and
b) via said at least one spiral inductor, applying electrical energy to the target tissue.

13. The method of claim 12, wherein:
each said spiral inductor comprises at least one spiral,
said spiral inductor is at least substantially planar,
said at least one spiral comprises an electrically conductive metal,
said spiral inductor includes an external surface,
said external surface of said spiral inductor defines a treatment face, and
said treatment face is configured for contacting the patient's body.

14. The method of claim 13, wherein:
each said spiral has a pitch in the range of from about 0.25 mm to 5 mm, and
each said spiral inductor comprises from about 10 to 200 turns.

15. The method of claim 12, wherein:
step a) comprises disposing said active electrode unit at a treatment area of the patient's body,
said active electrode unit is configured for selectively heating the target tissue relative to a non-target tissue,
step b) comprises heating the target tissue in the absence of actively cooling the non-target tissue,
the target tissue comprises subcutaneous fat, and
the non-target tissue comprises skin.

16. The method of claim 12, wherein:
said active electrode unit comprises a plurality of said spiral inductors,
said plurality of spiral inductors are arranged in an array such that said plurality of spiral inductors are at least substantially co-planar,
said apparatus is configured for sequentially energizing said plurality of spiral inductors, and
step b) comprises sequentially applying electrical energy to different areas of the target tissue via sequential energization of said plurality of spiral inductors.

17. The method of claim 12, wherein:
said active electrode unit includes a treatment face,
step a) comprises contacting the patient's skin with said treatment face, and
said treatment face comprises a bare metal external surface of said at least one spiral inductor.

18. The method of claim 12, wherein the target tissue comprises skin or subcutaneous fat of the patient.

19. A method for performing electrosurgery on a patient, comprising:
a) contacting the patient's body with a return electrode unit, wherein said return electrode unit comprises a spiral inductor;
b) applying electrical energy to the patient's body via an active electrode unit, wherein said active electrode unit is coupled to a power supply; and
c) receiving said electrical energy from the patient's body via said spiral inductor, wherein:
said return electrode unit includes a patient-contacting surface,
said spiral inductor comprises an electrically conductive metal, and
said patient-contacting surface comprises:
a patient-contacting layer having an electrical resistivity value less than 0.1 Ohm.m disposed on said spiral inductor, or
a bare metal surface of said spiral inductor.

20. The method of claim 19, wherein:
said patient-contacting surface comprises said bare metal surface of said spiral inductor,
said spiral inductor comprises at least one spiral of said electrically conductive metal,
said at least one spiral has a pitch in the range of from about 0.25 mm to 5 mm, and
each said spiral comprises from about 20 to 150 turns.

21. The method of claim 19, wherein:
said patient-contacting surface comprises said patient-contacting layer,
said spiral inductor comprises at least one spiral of said electrically conductive metal,
said spiral has a pitch in the range of from about 0.25 mm to 5 mm, and
said at least one spiral comprises from about 20 to 150 turns.

22. The method of claim 19, wherein:
said spiral inductor comprises a plurality of spirals of said electrically conductive metal,
said plurality of spirals are stacked vertically, and
each said spiral has the same spiral configuration, wherein:
each turn of a first spiral of said plurality of spirals is electrically coupled in series to a radially corresponding turn of each successive one of said plurality of spirals, and
each turn of an innermost spiral of said plurality of spirals is electrically coupled to an adjacent, radially outward turn of said first spiral, with the proviso that a radially outermost turn of said innermost spiral is not so coupled to an adjacent radially outward turn of said first spiral,
and wherein said first spiral is an outermost spiral of said plurality of spirals.

23. The method of claim 22, wherein:
said spiral inductor comprises from about two (2) to four (4) of said spirals, and
each said spiral comprises from about 10 to 200 turns.

24. The method of claim 19, wherein:
said spiral inductor is a return spiral inductor, and
said active electrode unit comprises an active spiral inductor.

25. A method for making a multi-layer spiral inductor, comprising:
a) forming a plurality of spirals, wherein each spiral comprises an electrically conductive metal disposed on an electrically insulating support layer;
b) stacking said plurality of spirals;
c) electrically coupling, in series, each turn of a first spiral of said plurality of spirals to a radially corresponding turn of each successive one of said plurality of spirals, and
d) electrically coupling each turn of an innermost spiral of said plurality of spirals to an adjacent, radially outward turn of said first spiral, and wherein said first spiral is an outermost spiral of said plurality of spirals, with the proviso that a radially outermost turn of said innermost spiral is not so coupled to an adjacent, radially outward turn of said outermost spiral.
